(19) United States
(12) Patent Application Publication
(10) Pub. No.: US 2026/0102239 A1
(43) Pub. Date: Apr. 16, 2026

(54) METHODS OF ESTABLISHING STEM CELL CULTURES FROM LOW-QUALITY EMBRYOS TO INCREASE THE RATE OF GENETIC PROGRESS

(71) Applicant: INGURAN, LLC, Navasota, TX (US)

(72) Inventors: Pablo Ross, Navasota, TX (US); Diego Andres Moreno Garcia, Nantes (FR)

(73) Assignee: INGURAN, LLC, Navasota, TX (US)

(21) Appl. No.: 19/264,208

(22) Filed: Jul. 9, 2025

Related U.S. Application Data
(60) Provisional application No. 63/669,155, filed on Jul. 9, 2024.

(51) Int. Cl. A61D 19/04; C12N 5/073; C12Q 1/6881

(52) U.S. Cl. CPC A61D 19/04; C12N 5/0604; C12Q 1/6881; C12Q 2600/124

(57) ABSTRACT

The invention includes a method of identifying high genomic value, non-human mammalian embryos that would otherwise be disposed of or frozen based on their morphology. The method generally involves establishing a stem cell culture from the non-human mammalian embryo, which entails generating a population of embryos in vitro or in vivo, assessing the morphology of each of the embryos in the population, selecting a subpopulation of embryos for embryo transfer based on the morphological assessment, thereby establishing a group of selected embryos and a group of unselected embryos in the population, and establishing a stem cell culture from an embryo in the group of unselected embryos.

### REFERENCE TO RELATED APPLICATIONS

This application claims the benefit of U.S. Provisional Patent Application No. 63/669,155, filed Jul. 9, 2024, the entire disclosure of which is incorporated herein by reference.

### BACKGROUND OF THE INVENTION

While assisted reproductive technologies, such as in vitro fertilization (IVF), as well as improved methods of phenotypic and genotypic evaluation, have allowed breeders to increase the rate of genetic progress in animals, such gains have been limited to date. At present, one limiting factor on the rate of genetic progress arises when embryos are either discarded or left unused due to poor morphology when assessed for suitability for transfer into a recipient. Embryos with poor morphology typically have lower pregnancy rates than those embryos with normal morphology, which may not be acceptable in many breeding programs. As a result, such embryos are often discarded prior to genomic evaluation. However, excluding such embryos from the pool of selection candidates reduces selection intensity and thus the rate of genetic progress in a breeding program all else being equal.

### SUMMARY OF THE INVENTION

One embodiment of the invention comprises a method of establishing a stem cell culture from a non-human mammalian embryo comprising generating a population of embryos, in vitro or in vivo; assessing the morphology of each of the embryos in the population; selecting a subpopulation of embryos for embryo transfer based on the morphological assessment, thereby establishing a group of selected embryos and a group of unselected embryos in the population; and establishing a stem cell culture from an embryo in the group of unselected embryos. In a further embodiment, the method further comprises a step of obtaining omics data from one or more cells from the stem cell culture. In yet another embodiment, the invention comprises a step of extracting DNA from one or more cells from the stem cell culture. In an even further embodiment, the invention comprises steps of genotyping the DNA extracted from the one or more cells from the stem cell culture to obtain a genotype and determining a genomic estimated breeding value (GEBV) or a genomic predicted transmitting ability (GPTA) based on the obtained genotype. In a yet further embodiment, the invention also comprises the step of producing a clone or a nuclear transfer egg using a cell from the stem cell culture; the step of transferring the clone into a recipient animal; the step of obtaining one or more embryonic or fetal cells from within an allantois of the transferred clone; the step of extracting DNA from the one or more embryonic or fetal cells; the step of genotyping the DNA extracted from the one or more embryonic or fetal cells to obtain a genotype for the clone; the step of determining a genomic estimated breeding value (GEBV) or a genomic predicted transmitting ability (GPTA) of the clone using the genotype; and the step of selecting the transferred clone as a parent or for production of gametes based on the determined GEBV or GPTA.

An additional embodiment of the invention comprises a method of establishing a cell culture from a non-human mammalian embryo comprising generating a population of embryos, in vitro or in vivo; assessing the morphology of each of the embryos in the population; selecting a subpopulation of embryos for embryo transfer based on the morphological assessment, thereby establishing a group of selected embryos and a group of unselected embryos in the population; and establishing a cell culture from an embryo in the group of unselected embryos using a media comprising an inhibitor or antagonist of the Wnt/β-catenin pathway. In a further embodiment, the method further comprises a step of obtaining omics data from one or more cells from the cell culture. In yet another embodiment, the invention comprises a step of extracting DNA from one or more cells from the cell culture. In an even further embodiment, the invention comprises steps of genotyping the DNA extracted from the one or more cells from the cell culture to obtain a genotype and determining a genomic estimated breeding value (GEBV) or a genomic predicted transmitting ability (GPTA) based on the obtained genotype. In a yet further embodiment, the invention also comprises the step of producing a clone or a nuclear transfer egg using a cell from the cell culture; the step of transferring the clone into a recipient animal; the step of obtaining one or more embryonic or fetal cells from within an allantois of the transferred clone; the step of extracting DNA from the one or more embryonic or fetal cells; the step of genotyping the DNA extracted from the one or more embryonic or fetal cells to obtain a genotype for the clone; the step of determining a genomic estimated breeding value (GEBV) or a genomic predicted transmitting ability (GPTA) of the clone using the genotype; and the step of selecting the transferred clone as a parent or for production of gametes based on the determined GEBV or GPTA. In a certain embodiment, the step of establishing a cell culture comprises placing the embryo in the media comprising an inhibitor or antagonist of the Wnt/β-catenin pathway. Another embodiment further comprises a step of passaging the cell culture 6 to 14, 6 to 8, or 7 days after placing the embryo in the media comprising an inhibitor or antagonist of the Wnt/β-catenin pathway

Another embodiment of the invention comprises a method of establishing a cell culture from a non-human mammalian embryo comprising generating an embryo, in vitro or in vivo; identifying the embryo as a low-quality embryo based on a morphological assessment of the embryo; and establishing a cell culture from the identified embryo using a media comprising an inhibitor or antagonist of the Wnt/β-catenin pathway. A further embodiment may also comprise the steps of obtaining omics data from one or more cells from the cell culture; extracting DNA from one or more cells from the cell culture; or genotyping the DNA extracted from the one or more cells from the cell culture to obtain a genotype and determining a genomic estimated breeding value (GEBV) or a genomic predicted transmitting ability (GPTA) based on the obtained genotype. This embodiment may further comprise a step of generating a clone of the embryo using a cell from the cell culture. A further embodiment may also comprise a step of identifying the clone as a low-quality embryo based on a morphological assessment of the clone. An even further embodiment of the invention may also comprise a step of establishing a cell culture from the clone using a media comprising an inhibitor or antagonist of the Wnt/β-catenin pathway. In a certain embodiment, the step of establishing a cell culture comprises placing the embryo in the media comprising an inhibitor or antagonist of the Wnt/β-catenin pathway. Another embodiment further comprises a step of passaging the cell culture 6 to 14, 6 to 8, or 7 days after placing the embryo in the media comprising an inhibitor or antagonist of the Wnt/β-catenin pathway

Yet another embodiment of the invention comprises a method of establishing a cell culture from a non-human mammalian embryo comprising identifying an embryo as a low-quality embryo based on a morphological assessment of the embryo; and establishing a cell culture from the identified embryo using a media comprising an inhibitor or antagonist of the Wnt/β-catenin pathway. A further embodiment may also comprise the steps of obtaining omics data from one or more cells from the cell culture; extracting DNA from one or more cells from the cell culture; or genotyping the DNA extracted from the one or more cells from the cell culture to obtain a genotype and determining a genomic estimated breeding value (GEBV) or a genomic predicted transmitting ability (GPTA) based on the obtained genotype. This embodiment may further comprise a step of generating a clone of the embryo using a cell from the cell culture. A further embodiment may also comprise a step of identifying the clone as a low-quality embryo based on a morphological assessment of the clone. An even further embodiment of the invention may also comprise a step of establishing a cell culture from the clone using a media comprising an inhibitor or antagonist of the Wnt/β-catenin pathway. In a certain embodiment, the step of establishing a cell culture comprises placing the embryo in the media comprising an inhibitor or antagonist of the Wnt/β-catenin pathway. Another embodiment further comprises a step of passaging the cell culture 6 to 14, 6 to 8, or 7 days after placing the embryo in the media comprising an inhibitor or antagonist of the Wnt/β-catenin pathway

In any of the above embodiments, a step of deriving a gamete from an embryo may comprise i) obtaining or deriving an embryonic stem cell from the embryo or ii) deriving an induced pluripotent stem cell from a fibroblast from the embryo.

The various embodiments of the invention may be applied to, or comprise, individuals or species of non-human mammals, and the invention should be understood not to be limited to the species of non-human mammals described by the specific examples within this application. Rather the specific examples within this application are intended to be illustrative of the varied and numerous species of non-human mammals to which the methods of the invention may be applied. Embodiments of the invention, for example, encompass animals having commercial value for meat or dairy production such as swine, ovine, bovine, equine, deer, elk, buffalo, or the like (naturally the mammals used for meat or dairy production may vary from culture to culture). They also encompass various domesticated non-human mammalian species such as canines and felines, as well as primates, including but not limited to, chimpanzees, and gorillas, as well as whales, dolphins and other marine mammals. In particular, embodiments of any of the above disclosed embodiments, the non-human mammalian species comprises bovids.

### BRIEF DESCRIPTION OF THE DRAWINGS

FIG. 1 is a diagram summarizing the steps of one embodiment of the invention.

### DETAILED DESCRIPTION OF THE INVENTION

The invention generally encompasses a method of expanding the pool of selection candidates from which a breeder can select parents of the next generation. In the context of a breeding program that utilizes embryo transfer, the invention allows one to genomically evaluate and select an embryo that would, using prior art methods, otherwise be discarded or not transferred into recipient female based on a morphological assessment of the embryo. By increasing the number of selection candidates in a breeding program, the rate of genetic progress is increased due to an increase in selection intensity, all else being equal.

Within a genetic nucleus, (or line or herd), once selected, parents that produce the next generation are mated with one another, while avoiding matings between closely related individuals, with the goal of increasing the genetic merit of the next generation. An increase in the genetic merit of the next generation constitutes genetic progress. An increase in genetic merit, in this context, means that for a given trait or set of traits, the individuals in the successive generation will express the desired trait or set of traits more strongly than their parents. With respect to undesirable traits, an increase in genetic merit means the individuals in the successive generation will express the trait or set of traits less strongly than their parents.

Genetic change, including desirable genetic change (i.e., genetic progress per year), (“dG”) can be measured as the difference between the average genetic level of all progeny born in one year and all progeny born the following year. The difference is the result of selected parents having higher genetic merit than the average genetic merit of all the selection candidates (the animals available for selection as parents of the next generation). In ideal conditions, this depends upon the heritability (h²) of the trait and the difference between the average performance of selected parents and that of selection candidates. The heritability of a trait (h²) is the proportion of observable differences (phenotypic variance, σ²_{P}) in a trait between individuals within a population that is due to additive genetic (A), as opposed to environmental (E), differences (h²=σ²_{A}/σ²_{P}=σ²_{A}/(σ²_{A}+σ²_{E})). The difference between the average performance of selected parents and that of all selection candidates (of which the selected parents are a subset) is also known as the selection differential.

The genetic progress per year is the result of genetic superiority of selected males and of selected females. This is expressed in the following equation:

$$dG = \{ {( {R}_{IH} * i )}_{males} + {( {R}_{IH} * i )}_{females} \} * {σ}_{H} / ( {L}_{males} + {L}_{females} ) ,$$

Where, R=the accuracy of selection, i=the selection intensity, σ_{H}=genetic variation and L=generation interval, for male or female parents.
H=breeding goal that combines genetic merit (g) of the traits (1 to m) that need to be produced weighted by the economic values (v) of the traits (H=v₁g₁+v₂g₂++vₘgₘ). The economic value is positive if selection is for larger phenotypic values and negative if selection is for smaller phenotypic values.
I=an index that combines all the trait information on the individual and its relatives and is the best estimate of the value of H for the individual.

As used herein, “breeding value” generally constitutes a value twice the average deviation of an animal's offspring group for a given trait from the population mean. A “parent average” generally constitutes the average of an animal's parents' breeding values, or the sum of the parents' predicted transmitting abilities (PTAs). “Genetic markers” generally constitute identifiable (i.e., typable) regions on the genome for which there is variation in the population. In Holstein dairy cattle breeding, for example, it is of importance to rank animals according to a breeding value. A genetic evaluation can be expressed as either a PTA or an estimated breeding value (EBV). Both are measures of performance relative to a base population, with an individual's PTA simply being one half of the EBV. A PTA indicates the difference in performance that can be expected from an animal's daughters relative to that base; an EBV is the genetic merit of the animal itself relative to the base and, therefore, is twice the PTA. Genomic EBVs (GEBVs) and genomic PTAs (GPTAs) are estimated breeding values and predicted transmitting abilities that incorporate genomic data, including genomic relationships, as commonly derived from single nucleotide polymorphism (SNP) data. As used herein, the terms “EBV” and “PTA” encompass GEBV and GPTA, respectively. The term “breeding value” as used herein encompasses EBVs. The term “genotypic value” encompasses an individual's breeding value plus non-additive genetic effects, such as dominance and epistasis.

In a large population, the selection intensity depends upon how many animals are tested and how many are selected—the lower the proportion selected the higher the selection intensity and the larger the genetic progress, all else being equal. Thus, in order to maximize genetic progress, one should rank all tested animals based on the GEBV, for example, and then select the minimum number of top males and females required to maintain the line, breed and/or herd size and to avoid inbreeding problems. This ensures that the average GEBV of selected animals is substantially higher than the average GEBV of all animals tested. In particular, through the use of artificial insemination (AI), one needs to select fewer males than females and the selection intensity for males is higher than for females.

The generation interval for males (or females) is the average age of male parents (or female parents) when progeny are born. The annual rate of genetic progress depends on the generation interval and on the superiority of the parent's GEBVs compared to that of the selection candidates. In general, males contribute more to the genetic progress per year than the females.

“Line” as used herein refers to animals having a common origin and similar identifying characteristics. “Genetic nucleus” as used herein refers to one or more populations of male and female animals used to generate selection candidates in a breeding program. “Breeding program” as used herein refers to a system for making genetic progress or for the creation of lines in a population of animals.

### Embryo Production In Vivo and In Vitro

In certain embodiments of the invention, embryos may be produced in vivo by traditional methods for synchronized supernumerary follicle production, artificial insemination (AI) and scheduled non-surgical transvaginal catheterized intrauterine embryo recovery. In other aspects of the invention, in vitro produced embryos may be produced in the laboratory by non-typical harvest of oocytes, in vitro fertilization (IVF) and embryo culture methodologies. In peripubertal heifers, prophase I immature cumulus oocyte complexes (COCs) are recovered from live standing females by using ultrasound guided transvaginal oocyte recovery (TVOR) system, also referred to as ovum pickup (OPU). In prepubertal heifers, ultrasound guided laparoscopic OPU is employed for COC recovery. When immature COCs are brought into the laboratory, they are placed into typical in vitro maturation (IVM) culture system where the most developmentally capable oocytes undergo spontaneous and programmed meiosis. After an overnight culture period, those oocytes that progress through meiosis I (and accordingly shed their second polar body progressing to metaphase of the second meiotic division) and are morphologically normal (including an intact plasma membrane) are used in IVF. Mature oocytes from individual females are placed into traditional IVF drops and mated to specific sires, using highly screened and accurate sperm capacitation treatments and sperm concentration per oocyte fertilized.

In certain embodiments of the invention, zygotes (day 1) are placed into traditional co-culture system and cultured to uterine stages of development by day 7 to 8 of culture. Embryos are typically transported to a recipient heifer farm where they are non-surgically transferred. Prior to transfer, embryos may be biopsied or sampled for genetic screening and/or genomic evaluation. Within certain specific stages of embryo development, embryos can be dismantled and used in embryo multiplication procedures and/or cryopreserved for later use. Embryos destined for transfer to synchronized surrogate females are transported to the farm in culture and non-surgically transferred by traditional methods. In certain embodiments, the invention contemplates that recipient females are regularly checked by veterinarians and ongoing pregnancies are monitored on a regular and scheduled basis via transrectal real time ultrasonography.

### Morphological Assessment of Embryos

In the context of the invention, the term “morphology” with respect to an embryo means the form or structure of the embryo. Accordingly, with respect to an embryo of the invention, the phrase “assessing the morphology” means evaluating the form or structure of the embryo, which includes but is not limited to 1) evaluating the absolute or relative size, shape, color, symmetry, location or volume of structural features of the embryo, including but not limited to the perivitelline space, the zona pellucida, the trophoectoderm, the inner cell mass, the blastocoele cavity and blastomeres comprising the embryo or 2) evaluating the stage of development of the embryo. Similarly, with respect to an embryo of the invention, the phrase “morphological assessment” means an 1) evaluation the absolute or relative size, shape, color, symmetry, location or volume of structural features of the embryo, including but not limited to the perivitelline space, the zona pellucida, the trophoectoderm, the inner cell mass, the blastocoele cavity and blastomeres comprising the embryo or 2) evaluation of the stage of development of the embryo.

For embryo transfer, the morphology of an embryo prior to transfer into a recipient is important since it influences the viability and development of the embryo post-transfer. In particular, assessing the morphology of an embryo prior to transfer tells one the stage of development and the quality of the embryo, with underdeveloped or low-quality embryos generally suffering reduced viability post-transfer.

Morphological assessment of embryos according to International Embryo Technology Society (IETS) standards (see, e.g., Manual of the International Embryo Transfer Society, 4th Edition, 2009) is generally accepted practice in commercial and research settings. In one embodiment of the invention, IETS standards can be used to assess the morphology of an embryo. Morphological assessment of an embryo under IETS standards requires that an embryo be evaluated for both its stage of development and its quality. Accordingly, in one embodiment of the invention, once the stage of development and the quality of the embryo are assessed, one can assign a quality score to the embryo.

In one embodiment of the invention, the stages of embryo development that can be considered in grading an embryo are as follows:
Stage 1: Unfertilized Oocyte
Stage 2:2 to 16 cell Embryo
Stage 3: Early or Pre-compaction Morula—The blastomeres of the embryo are not yet compacted.
Stage 4: Morula—The blastomeres of the embryo are compacted so that the individual cells of the embryo are unable to be resolved or counted. Additionally, at this stage, peri-vitelline (PV) space encompasses the embryo and the thickness of the zona pellucida (ZP) is similar to earlier cleavage stages of the embryo.
Stage 5: Early blastocyst—The blastocoele cavity takes up less than half of the volume of the embryo proper. Additionally, PV space is still evident around the embryo proper and may or may not touch the ZP.
Stage 6: Blastocyst—The blastocoele cavity takes up at least half of the volume of the embryo proper. Small amounts of PV space are present, and the thickness of the ZP remains unchanged from earlier stages of development.
Stage 7: Expanded blastocyst—The embryo proper takes up all of the space under the zona pellucida, and no PV space remains. The volume of the blastocoele cavity is greater than that of the inner cell mass (ICM), which should be compact and identifiable. The ZP is evident but thinner compared to earlier stages of development. Expanded blastocysts may vary in size depending on expansion of the blastocoele cavity. An embryo that has begun expansion and is observed to have a thinning ZP may be considered stage 7. Embryos generated in vitro or in vivo can be observed to collapse and re-expand during the course of the blastocoele expansion and hatching phases of development. However, any collapse should not change the stage classification. With respect to collapsed embryos, the thickness of the ZP and diameter of the embryo (including the ZP) should be used as the main indicator of stage.
Stage 8: Hatching blastocyst—The ZP has thinned, and the trophectoderm (TE) may be appear to be herniating through an opening in the ZP. The ICM has further compacted and is readily identifiable. Regardless of progression of herniation (including no herniation), all embryos with an opening in the ZP in which the embryo proper is still in contact with the ZP is considered hatching. Some IVF embryos may be observed to hatch without thinning of the ZP, which may be due to zona hardening or from damage sustained during manipulation.
Stage 9: Hatched blastocyst—The blastocyst has completely emerged from the ZP. The TE will appear bumpy without the presence of ZP, and the ICM is compact and readily identifiable.

In addition to assessing the stage of embryo development, in one embodiment of the invention, a morphological assessment can also include assessing the quality of an embryo. In a particular embodiment of the invention, the following categories describing the quality of an embryo can be used.

Code 1: Excellent or good quality—The embryo has a symmetrical and spherical embryo mass with blastomeres that are uniform in size, color, and density. For embryos generated in vivo, at least 85% of the cellular material contained within the ZP should be part of the embryo proper or viable embryonic mass, which should be determined based on the percentage of material in the PV space relative to the embryo proper. For embryos at the early blastocyst stage of development and later, the ICM and TE should be readily discernable. Trophectoderm cells should be relatively uniform in size and light in color. The ICM should become increasingly compact as embryo development advances. Embryos with minor deformities of the ZP and with no other deficiencies may be classified as grade 1.

Code 2: Fair quality—There are moderate irregularities in overall shape of the embryonic mass or in size, color, and density of individual cells within the mass. At least half of the cellular material should be part of an intact, viable embryonic mass. Morula with poor compaction present in part of the embryo proper should be downgraded to a 2. Minor defects in one of either the ICM or TE are acceptable, but not in both. Defects of the ICM may include poor organization, poor compaction, or speckled or dense appearance of individual cells within the mass. Defects of the TE may include cells with uneven size, clustered or scattered excessively dense cells, or irregularities in the shape of the blastocoele cavity.

Code 3: Poor quality—There are major irregularities in shape of the embryonic mass or in size, color, and density of individual cells within the mass, and at least 25% of the cellular material should be part of the intact, viable embryonic mass. Major defects are present in both the ICM and TE. Poor quality embryos may be lacking an readily discernable ICM or may have very few cells of varying densities that comprise a poorly organized ICM. The TE may have few cells, with many of those cells larger than normal.

Code 4: Degenerated—Unfertilized ova and embryos that have stopped developing at cleavage stages are considered non-viable if present 7 days after IVF. Blastomeres may appear grainy, and cytoplasm may be separating from the plasma membrane. Additionally, no viable embryonic mass is identifiable.

In one embodiment of the invention, embryos that are not developing at the expected rate are typically downgraded. When generating embryos via in vitro fertilization, “Day 0” means the time at which co-incubation of sperm and ova is initiated. In contrast, when generating embryos in vivo (such as by uterine flushing), “Day 0” is defined by standing estrus in the donor cow (ovulation and fertilization may not occur for another 12 to 24 hours); and seven days after standing estrus, embryo recovery may yield embryos ranging from stage 4 to 7. The majority of fertilization events will occur within 12 hours of the beginning of co-incubation, and a final evaluation of an embryo generated via IVF will be made 6 or 7 days after fertilization. Thus, on a day-by-day comparison, embryos generated via IVF compared to embryos generated in vivo are more advanced.

In one embodiment, with respect to embryos generated via IVF, Day 3 embryos (72 h after IVF) should have at least 4 blastomeres; Day 6 embryos should be at Stages 4 and 5; Day 7 embryos should be at Stage 6 and preferably Stage 7 (and sometimes 8 or 9). In one embodiment, a morula on Day 7 is considered a grade 2 embryo because it is delayed 12 to 24 hours in development.

In one embodiment of the invention, the quality score (or grade) that should be assigned to a morphologically normal embryo evaluated on Day 7 will differ between an embryo produced via IVF and an embryo produced in vivo even when the stage of development is the same. For example, an embryo that is at Stage 3 on Day 7 should receive a quality score of 3 if it was generated via IVF and a quality score of 2 if it was produced in vivo; an embryo that is at Stage 4 on Day 7 should receive a quality score of 2 if it was generated via IVF and a quality score of 1 if it was produced in vivo; an embryo that is at Stage 5 on Day 7 should receive a quality score of 1 or 2 if it was generated via IVF and a quality score of 1 if it was produced in vivo; an embryo that is at Stage 6 on Day 7 should receive a quality score of 1 if it was generated via IVF and a quality score of 1 if it was produced in vivo; and an embryo that is at Stage 7 on Day 7 should receive a quality score of 1 if it was generated via IVF and a quality score of 1 if it was produced in vivo. In one embodiment of the invention, a stereomicroscope with at least 50×magnification can be used to assess the morphology of an embryo.

In certain embodiments, embryos may be morphologically assessed 7 days after fertilization (i.e., Day 7 with respect to embryos generated in vitro). In other embodiments of the invention, embryos may be removed from culture earlier (e.g., Day 6) in order to avoid embryo hatching prior to embryo transfer or cryopreservation.

For purposes of the invention, the phrase “low-quality embryo” means an embryo that 1) has one or more of the morphological features listed above for Code 2, 3 or 4 category embryos or 2) by Day 7 (i.e., 7 days after co-incubation of sperm and egg in the case of embryos generated in vitro or 7 days after standing estrus of the female donor animal in the case of embryos generated in vivo), has not reached the morula stage of development if produced in vivo or the early blastocyst stage of development if produced in vitro.

Generating Pluripotent Stem Cell Cultures from Embryos after Morphological Assessment

One embodiment of the invention comprises morphologically assessing an embryo, selecting the embryo for culturing based on the morphological assessment, and culturing the embryo to generate a pluripotent stem cell culture. A more particular embodiment of the invention encompasses a method for generating pluripotent stem cells from an embryo that would otherwise remain unselected for embryo transfer based on a morphological assessment of the embryo. In an even more particular embodiment of the invention, a hatched embryo that would otherwise remain unselected for embryo transfer based on a morphological assessment of the embryo is used to establish a pluripotent stem cell culture. Alternatively, for an embryo that is not hatched (e.g., an expanded blastocyst), the zona pellucida can be mechanically or chemically removed. Mechanical removal of the zona pellucida from an embryo can be accomplished in any suitable embryo splitting media using a needle or a laser, for example. Alternatively, the zona pellucida can be chemically removed, using pronase for example. In a particular embodiment of the invention, a 21 G needle is used for mechanical separation of the zona pellucida from an embryo.

In one embodiment of the invention, once denuded, the embryo is placed into a culture dish or well that is coated with mitotically inactivated feeder cells. In a particular embodiment of the invention, the inactivated feeder cells are comprised of irradiated mouse fibroblasts, and in an even more particular embodiment, are comprised of CF1 mouse embryonic fibroblasts (MEFs). In an alternative embodiment, the embryo is placed into a culture dish free of feeder cells. In certain embodiments of the invention, a culture dish free of feeder cells can be coated with a cell support material, for example, fibronectin, laminin, vitronectin or Matrigel (Corning Life Sciences, Tewksbury, MA) or a combination thereof.

The embryos are then cultured in a serum-free media comprising an inhibitor or antagonist of the Wnt/β-catenin pathway and either 1) a fibroblast growth factor (FGF) or 2) a member of the transforming growth factor beta (TGF-β) family or both. Examples of inhibitors of the Wnt/β-catenin pathway that may be used in the invention include, but are not limited to, inhibitors of Wnt response (IWR) including IWR1, IWR2, IWR3, IWR4 and IWR5 and inhibitors of Wnt production (IWP) including IWP1, IWP2, IWP3 and IWP4 and may be used a concentration of 0.5-5 μM, 1-4 μM, 2-3 μM or 2.5 μM. Examples of fibroblast growth factors that may be used in the invention include, but are not limited to, FGF1, FGF2, FGF3 and FGF4 and may be used at a concentration of 10-30 ng/ml, 15-25 ng/ml, 17-23 ng/ml or 20 ng/ml. Examples of members of the TGF-β family that may be used, include but are not limited to TGF-β1, Activin A and Activin B and may be used at a concentration of 10-30 ng/ml, 15-25 ng/ml, 17-23 ng/ml or 20 ng/ml. In a particular embodiment of the invention, the serum free media is comprised of an IWR and either 1) FGF2 or 2) Activin-A or both. In a more particular embodiment of the invention, the serum free media is comprised of IWR1 and FGF2. In another embodiment of the invention, the serum free media is comprised of IWR1, FGF2 and Activin A. In one embodiment of the invention, the serum-free media is comprised of TeSR-E6 (Stem Cell Technologies, Canada), although any serum-free media suitable for use in stem cell culturing may be used as a base media in the invention. In a particular embodiment of the invention, the serum-free media is supplemented with a serum protein, for example bovine serum albumin (BSA) at a concentration of 10-20 mg/ml, 12-17 mg/ml, 13-15 mg/ml or 13.4 mg/ml. In particular, embodiment of the invention, the BSA is comprised of a low free fatty acid BSA. The media in which the pluripotent stem cells are cultured may also comprise a suitable antibiotic, including but not limited to penicillin or streptomycin. In a particular embodiment of the invention, the serum free culture media comprises IWR1, FGF2, Activin A and BSA. In another embodiment, the serum free culture media comprises IWR1, FGF2 and Activin A. In a more particular embodiment, the serum fee culture media comprises TeSR-E6, IWR1 and FGF2; TeSR-E6, IWR1, FGF2 and BSA; TeSR-E6, IWR1, FGF2 and Activin A; or TeSR-E6, IWR1, FGF2, Activin A and BSA. In a particular embodiment of the invention, the serum-free media may be comprised of Dulbecco's Modified Eagle Medium: Nutrient Mixture F-12 (DMEM/F-12), examples of which include TeSR1 media disclosed by Ludwig et al. (Nature Methods, vol. 3, no. 8, August 2006, pp. 637-646); modified mTeSR1 media as disclosed by Wu et al. (Nature, 2015 May 21; 521 (7552): 316-321), which lacked FGF2 and TGFß1 but was supplemented with 20 ng/ml FGF2 and 2.5 μM IWR1; and N2B27 medium as disclosed by Tong et al. (“Generating gene knockout rats by homologous recombination in embryonic stem cells,” Nat Protoc. 2011 June; 6 (6)). In a particular embodiment of the invention, pluripotent stem cells can be cultured using a culture dish free of feeder cells and a serum-free media comprising FGF2, IWIR1 and Activin A. In an even more particular embodiment, pluripotent stem cells can be cultured using a culture dish free of feeder cells and a serum-free media comprising FGF2, IWIR1, Activin A and BSA.

Once the embryo is placed in a suitable culture media as above, the embryo is cultured at approximately 30-40° C., 35-39° C., 36-38° C., or 37° C. and with approximately 5% CO₂. After approximately 50-100 hours, 60-90 hours, 70-80 hours, or 72 hours, the culture media is replaced. If an embryo has failed to attach to the layer of feeder cells in the culture at the time the culture media is replaced, the embryo can be mechanically pressed against the bottom of the culture dish, using for example, a needle. After approximately 7 to 10 days (or alternatively, 6 to 14 days, 6 to 8 days, 11 to 14 days, or 7 days) in culture, the whole outgrowth is passed to a new culture dish or well coated with inactivated feeder cells, and the composition of the media used to establish the initial cell culture may be used again in the replacement media. In certain embodiments, outgrowths may be dissociated using trypsin or TrypLE (Gibco, Thermo Fisher Scientific, Waltham, MA). In a particular embodiment, the media used to replace the initial cell culture media is supplemented with an inhibitor of Rho-associated, coiled-coil containing protein kinase (ROCK) at a concentration of 1-20 μM, 3-17 μM, 5-15 UM or 10 μM. In a particular embodiment, inhibitors of ROCK with which the media can be supplemented, include but are not limited to, Y-27632 and fasudil. In an even more particular embodiment, the media used to replace the initial culture media is supplemented with Y-27632. In a particular embodiment, ROCK inhibitor (for example Y-27632) is used in the culture media for 24 hours, and after 24 hours, the culture media is replaced with a culture media that does not comprise a ROCK inhibitor (for example Y-27632). On the day of the first cell passage (e.g., at day 4, 5, 6, 7, 8, 9, 10, 11, 12, 13, 14, 15 or days 4 to 7, 6 to 14, 6 to 8, 7 to 10, 10 to 15, or 15 to 20, after seeding or at any passage thereafter, omics data may be obtained or extracted from for the cultured cells using a portion of the dissociated (i.e., unbound) cells or the culture media. In a more particular embodiment, dissociated cells may be genomically evaluated, by for example, genotying with a SNP microarray or by DNA sequencing.

One embodiment of the invention, provided by way of example only, includes the following method.
1. Culture IVF embryos, as above, in a serum-free media comprising an inhibitor or antagonist of the Wnt/β-catenin pathway.
2. 24 hours later, embryos that have not attached to the bottom of the well are pressed to the bottom of the well with a needle.
3. 24 hours later, viable embryo cultures—i.e., embryos that attach to the bottom of the culture well and produce an outgrowth—are selected.
4. 7 days after putting the embryos into culture, the cultures that produce a large and consistent outgrowth are selected. Half of the outgrowth is sent for genomic evaluation (e.g., via genotyping), while the culture process continues. (Typically, none of the cultures have produced embryonic stem cells by this time.)
5. Typically, after the second passage, the cell cultures begin showing the presence of embryonic stem cells.
6. When the genomic evaluation results arrive, a selection of embryos is made-if the results show low genetic merit for an embryo, the culture is discarded even if it has already become an embryonic stem cell culture. Alternatively, if the results show high genetic merit for an embryo, one or more cells from the cell culture are used as donors for generating one or more clones, regardless of whether the cell culture is comprised of embryonic stem cells yet or not.
7. Clones once generated are vitrified for embryo transfer.
8. If a high genetic merit embryo fails to successfully produce an embryonic stem cell culture, or if its clones are low-quality embryos unsuitable for embryo transfer, one or more of the low-quality clones can be placed back into culture, as above, and the process repeated until an embryonic stem cell culture is successfully established or a morphologically normal or high-quality embryo is produced.
   Estimating Production Values, Genotypic Values or Breeding Values from Omics Data (Including Genomic, Transcriptomic and Metabolomic Data)

In addition to estimating breeding values from genomic data, one embodiment of the invention encompasses estimating production, genotypic or breeding values from omics data generally. “Omics data” may include, but is not limited to, genomic, proteomic, transcriptomic, epigenomic, microbiomic or metabolomic data. Omics data is believed to take into account complex epistatic interactions that are not necessarily captured by genomic data alone. In the context of the invention, a “breeding value” is comprised of the sum of all gene effects that are relevant for a particular trait; a “genotypic value” is comprised of the breeding value, plus all gene interaction effects (i.e., dominance and epistasis); finally, a “production value” is comprised of the genotypic value plus the permanent environmental effects for the individual, including constant features.

In one embodiment of the invention, omics data is derived or obtained from molecules (small or large) or any other substances (ions, elements, etc.) obtained or extracted from an embryonic or fetal cell, including a stem cell or pluripotent stem cell, tissue sample or allantoic or amniotic fluid, or detected in the cell, tissue sample or allantoic or amniotic fluid. Both the presence and the quantity of such molecules or substances within a sample may be determined. Any known method in the art for detecting, measuring, quantifying or assaying molecules or other substances may be used with the invention, including but not limited to molecular hybridization, immunohistochemistry, real time quantitative PCR, quantitative reverse transcription PCR, blotting, nucleotide sequencing, protein sequencing, nuclear magnetic resonance spectroscopy, mass spectroscopy, liquid chromatography, gas chromatography and electrophoresis. In a specific embodiment, a transcriptome may be profiled using a microarray.

In a particular embodiment, transcriptomic, proteomic or metabolomic data can be derived from RNA, proteins or metabolites, respectively, found within a cell, tissue or allantoic fluid sample. In certain embodiments, a cell or tissue sample may be obtained from allantoic fluid in accordance with any of the methods described hereinabove. Such a cell sample may be cryopreserved and then subsequently thawed for extraction of DNA or RNA or to obtain proteins or metabolites for profiling or any molecules providing omics data.

In one embodiment of the invention, omics data comprises features. For example, for metabolomic data, each assayed or measured metabolite can constitute a feature. In one embodiment, a feature may simply comprise the presence or absence of a particular molecule or substance, e.g., the presence of a particular metabolite or transcript, or alternatively a feature may comprise the quantity of a particular molecule or substance, e.g., the quantity of a particular metabolite or transcript. For example, the quantity of glucose in a tissue or blood sample can comprise a feature.

These features from the omics data can be entered into a training model in which feature weights are obtained or estimated. Any suitable training model known in the art may be used with the invention. See for example, Westhues et al., “Omics-based hybrid prediction in maize,” Theor. Appl. Genet. (2017) 130:1927-1939; Sharifi-Noghabi et al., “MOLI: multi-omics late integration with deep neural networks for drug response prediction,” Bioinformatics (2019) 35:1501-i509; and Kim et al., “Multi-omics integration accurately predicts cellular state in unexplored conditions for Escherichia coli,” Nature Communications (2016), DOI 10.1038/ncomms13090, pages 1-12. For example, the normalized relative quantity of metabolites or mRNA can form feature blocks. Every metabolite or mRNA may be used as one distinct feature that contributes to the prediction of the variable or trait of interest.

Generally, the phenotype or trait can be modelled as a function of the feature set:

$$y = f ( z ) + e ,$$

where f( ) is any conceivable linear or non-linear function of the feature set in z that maps to y and e are the residuals.

One such function is a linear mixed model, in which a linear combination of feature covariables and weights result in the predicted phenotype:

$$y = Xb + Zu + e ,$$

where X is an incidence matrix for fixed effects (intercept and structural components of a potential trial design), Z is a matrix of feature covariates and u a vector of feature weights.

The predicted phenotype or traits is then: ŷ=: Zu.

These feature weights can then be used downstream for the prediction or calculation of production values, genotypic values or breeding values for animals or cell or tissue samples. For example, the variable or trait of interest that enters the training model may be breeding values. The predicted value using the feature weights will then also be a breeding value by design. The same is true for production of genotypic values. A production value can be predicted by using the raw phenotypic observations as dependent variables while employing the available features for the prediction of that phenotype.

With respect to genomic data, in various embodiments of the invention, genomic data may comprise DNA or RNA-related data obtained from oligonucleotide arrays or other hybridization assays, DNA sequence data or RNA sequence data. In a specific embodiment of the invention, genomic data may be obtained from whole or partial genome sequencing using any technique known in the art. In addition to obtaining genomic DNA sequences, in other embodiments of the invention, RNA may also be sequenced, including messenger RNA (mRNA), precursor mRNA (pre-mRNA), transfer RNA (tRNA), ribosomal RNA (rRNA), non-coding RNA (ncRNA), long RNA, including long non-coding RNA (lncRNA) and small RNA, including micro RNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). In addition to sequencing such molecules, it is also contemplated that real time quantitative PCR or quantitative reverse transcription PCR may be used to quantify DNA or RNA in a sample.

One embodiment of the invention therefore comprises a method of determining a production value, a genotypic value or a breeding value of a non-human mammalian embryo or fetus comprising obtaining omics data comprising one or more features from one or more embryonic or fetal cells (including but not limited to stem cells and pluripotent stem cells); calculating feature weights for the one or more features; calculating a production value, a genotypic value or a breeding value of the embryo or the fetus based on the calculated feature weights; selecting the embryo or fetus as a parent or to produce gametes based on the calculated production value, genotypic value or breeding value; and producing offspring from the selected embryo or fetus.

### DNA Extraction and Amplification

Another aspect of the invention encompasses sequencing or genotyping embryonic or fetal cells (including but not limited to stem cells and pluripotent stem cells). In a specific embodiment, the DNA of cultured pluripotent stem cells can be used for sequencing or genotyping. Pluripotent stem cell DNA may first be extracted and then amplified (via PCR) so that there is a sufficient amount of DNA for sequencing or genotyping.

For genomic analysis, approximately 200 ng of double stranded DNA should be extracted per sample DNA at concentration per sample of 50 ng/μl. In certain embodiments of the invention, the DNA is used to confirm the presence of a lethal haplotype and/or confirm that the lethal haplotype has been corrected or suppressed via genetic modification. The remaining cells in culture remain in cell culture for passage and eventual harvest and cryopreservation for later diagnostic, cytogenetic and biological productive use such as gene editing and cloning.

By way of example, the following DNA extraction and amplification procedure may be used in certain embodiments of the invention. One skilled in the art will know that variations on this method exist and that this method should not be construed to limit the functionality or scope of the current invention. This method is illustrative only.

1.5 ml tubes containing a cell suspension of pluripotent stem cells are spun at ≥10000×g in a microcentrifuge for 45 seconds to pellet the cells. The suspension solution is pipetted off carefully so as to not remove the pelleted cells. Approximately 50 μl of suspension solution is left in each tube. The tubes are then vortexed for 10 seconds to resuspend the cell pellets. 300 μl of Tissue and Cell Lysis Solution (Epicentre; Madison Wisconsin; Catalog #MTC096H) containing 1 μl of Proteinase K (Epicentre; Madison Wisconsin; at 50 μg/μl; Catalog #MPRK092) is then added to each tube and mixed. The tubes are incubated at 65° C. for 30 minutes and vortexed at 15 minutes. The samples are cooled to 37° C. Afterwards 1 μl of 5 mg/μl RNase A (Epicentre; Madison Wisconsin; at 5 mg/ml; Catalog #MPRK092) is added to each sample and then mixed. The samples are then incubated at 37° C. for 30 minutes. The samples are then placed in a 4° C. cooler for 5 minutes. 175 μl of MPC Protein Precipitation Reagent (Epicentre; Madison Wisconsin; Catalog #MMP095H) is added to each sample, and the samples vortexed vigorously for 10-15 seconds. The samples are centrifuged in order to pellet debris for 8 minutes at ≥10000×g. The supernatant is transferred to a clean microcentrifuge tube. 600 μl of cold (−20° C.) isopropanol is added to the supernatant. Each tube is then inverted 30-40 times. The DNA is pelleted by centrifugation for 8 minutes in a microcentrifuge at ≥10000×g. The isopropanol is poured off without dislodging the DNA pellet. The pellet is rinsed once with 70% ethanol and then the ethanol is carefully poured off so as not to disturb the DNA pellet. The residual ethanol is removed with a pipet, and the DNA pellet is allowed to air dry in the microcentrifuge tube. Once dried, the DNA pellet is resuspended in 20 μl Tris-EDTA.

In certain embodiments of the invention, DNA from pluripotent stem cells can be extracted using the Purelink Genomic Kit Cat #K1820-00 (Invitrogen). In further embodiments, once the DNA is extracted, it can be put through a whole genome amplification protocol using the Illustra Genomiphi V2 DNA amplification kit (GE Lifesciences), which uses the phi29 DNA polymerase to amplify the genome.

### Genotyping DNA

In one aspect of the invention, extracted and/or amplified DNA from stem cells may be genotyped using genomic single nucleotide polymorphism (SNP) arrays or chips, which are readily available for various species of animals from companies such as Illumina and Affymetrix. Alternatively, the entire genome can be sequenced using methods well-known in the art. Low density and high density chips are contemplated for use with the invention, including SNP arrays comprising from 3,000 to 800,000 SNPs. By way of example, a “50K” SNP chip measures approximately 50,000 SNPs and is commonly used in the livestock industry to detect lethal haplotypes and to establish genetic merit or genomic estimated breeding values (GEBVs).

Alternatively, nucleotide sequencing can be used in the invention to genotype DNA. One aspect of the invention comprises nucleotide sequencing extracted DNA or RNA. In certain embodiments of the invention, nucleic acid is extracted from pluripotent stem cells using any known method known in the art, including but not limited to Sanger sequencing and high throughput sequencing, which includes next generation (short read) sequencing and third generation (long read) sequencing. In one embodiment of the invention, one read with short read sequencing comprises approximately 100 to 300 base pairs, and one read with long read sequencing comprises approximately 10,000 or more base pairs. Nonlimiting examples of sequencing methods for use in the invention include single-molecule real time sequencing, ion semiconductor sequencing, pyrosequencing, sequencing by synthesis, combinatorial probe anchor synthesis, sequencing by ligation, nanopore sequencing, massively parallel signature sequencing, polony sequencing, DNA nanoball sequencing, heliscope single molecule sequencing and sequencing using droplet based microfluidics or digital microfluidics.

### Determining GEBVs Using Genetic Markers

The basis, and algorithm, for using genetic markers in determining GEBVs is found in Meuwissen et al., “Prediction of total genetic value using genome-wide dense marker maps,” Genetics 157, 1819 1829 (2001), which is incorporated by reference herein in its entirety. Implementation of genomic data in predictions for desirable traits is found in Van Raden, “Efficient Methods to Compute Genomic Predictions,” Dairy Science 91, 4414 4423 (2008), which is incorporated by reference herein in its entirety.

Livestock in the United States are often ranked using selection indexes that incorporate data related to various commercially important traits. With the advent of genomic testing, genomic data is now commonly used to predict these traits. To calculate an animal's score for a genomic selection index, one must first calculate the animal's GEBVs for each trait in the index, which can be accomplished using the teachings in Meuwissen et al. and VanRaden, above. Next, one determines the economic weight for each trait in the index. Finally, to determine the animal's score for the selection index, multiply each trait's GEBV by its economic weight and then sum all of these values together.

A genomic index commonly used in the United States for dairy cattle is the Genomic Total Performance Index (GTPI®), which is comprised of the following traits: protein; feed efficiency; dairy form; feet and legs composite; somatic cell score; daughter calving ease; fat; udder composite; productive life; fertility index; and daughter stillbirth. In certain embodiments, feed efficiency is equal to the dollar value of milk produced less feed costs for extra milk and less extra maintenance costs, and the fertility index is a function of heifer conception rate, cow conception rate and daughter pregnancy rate. In other embodiments of the invention, GEBV is used to determine Genomic Predicted Transmitting Ability (GPTA).

By way of example, in addition to determining a GEBV for a trait, the presence or absence of any of the following diseases and/or traits can be detected using SNP data or genomic data: Demetz syndrome; white heifer disease; Weaver syndrome (haplotype BHW); haplotype HHD; haplotype HH1; lethal brachygnathia trisomy syndrome; haplotype HH0; bovine hereditary cardiomyopathy; bovine dilated cardiomyopathy; neuronal ceroid lipofuscinosis; bovine chondrodysplastic dwarfism; notched ears/nicked ears; idiopathic epilepsy; bilateral convergent strabismus with exophthalmos; haplotype BHP; haplotype HHP; haplotype JHP; neuropathic hydrocephalus/water head; congenital hypotrichosis and anodontia defect/ectodermal dysplasia; ichthyosis fetalis; lethal trait A46/bovine hereditary zinc deficiency; Marfan Syndrome; double muscling; multiple ocular defects; bovine ocular squamous cell carcinoma; pink tooth; posterior paralysis/hind-limb paralysis; haplotype BHM; bovine spongiform encephalopathy/mad cow disease; mule foot disease (haplotype HHM); myophosphorylase deficiency; dropsy; black/red coat color (haplotype HBR; haplotype HHR); BAND3 deficiency; Charolais ataxia; bovine spinal dysmyelination (haplotype BHD); Dun coat color in Dexter cattle; bovine familial convulsions and ataxia; bulldog calf; simmental hereditary thrombopathy; GHRD; renal tubular dysplasia (RTD)/chronic interstitial nephritis; Hereford white face; haplotype HHC; developmental duplications; black kidney; cardiomyopathy/Japanese black cattle; crooked tail syndrome; congenital pseudomyotonia; bovine hereditary arthrogyposis multiplex congentia; belted; Syndrome d′Hypoplasie Généralisée Capréoliforme; fawn calf syndrome; bovine neonatal pancytopenia; rat-tail syndrome; cheilognathoschisis; German White Fleckvieh syndrome; haplotype JH1; paunch calf syndrome; acorn calf disease/congenital joint laxity and dwarfism; haplotype HH2; haplotype HH3; haplotype HH4; Holstein bull-dog dwarfism; haplotype AH1; haplotype HH5; haplotype JH2; and lethal arthrogyposis syndrome.

### Selection of Embryos as Parents of the Next Generation in a Breeding Program

One aspect of the invention encompasses selecting one or more embryos-rather than reproductively mature animals—as parents of the next generation in a breeding program. One embodiment involves selecting embryos as parents based on one or more breeding values, including EBVs and GEBVs, or genotypic values, of the embryo. For example, in a particular embodiment of the invention, a plurality of embryos is generated, and then each embryo is genomically evaluated and ranked relative to the other embryos on the basis of genetic merit. Embryos with the highest genetic merit may be selected as parents. In addition to selecting embryos on the basis of EBVs or genotypic values, one embodiment of the invention also includes selecting an embryo based on its gamete variance, as described in PCT/US2018/040353, the disclosure of which is hereby incorporated by reference herein.

### Embryo Transfer

Certain embodiments of the invention encompass embryo transfer. The following surgical and non-surgical methods of embryo transfer are provided by way of non-limiting example only.

In cattle, an embryo can be transferred via mid-line abdominal incision, or a flank incision, to a recipient under general anesthesia. Recipients are placed in squeeze chutes that give access to either flank. The corpus luteum is located by rectal palpation and the flank ipsilateral to the corpus luteum is clipped, washed with soap and water, and sterilized with iodine and alcohol. About 60 ml of 2 percent procaine is given along the line of the planned incision. A skin incision is made about 15 cm long, high on the flank, just anterior to the hip. Muscle layers are separated, and the peritoneum is cut. The surgeon inserts a hand and forearm into the incision, locates the ovary, generally about 25 cm posterior to the incision, and visualizes or palpates the corpus luteum. The uterine horn is exteriorized by grasping and stretching with the thumb and forefinger the broad ligament of the uterus, which is located medial to the uterine horn. A puncture wound is made with a blunted needle through the wall of the cranial one-third of the exposed uterine horn. Using about 0.1 ml of medium in a small glass pipette (<1.5 mm outside diameter), the embryo is drawn up from the storage container. The pipette is then inserted into the lumen of the uterus, and the embryo is expelled. The incision is then closed, using two layers of sutures.

Alternatively, a non-surgical method may be used to transfer an embryo in cattle. First, it is necessary to palpate ovaries in order to select the side of ovulation, since pregnancy rates are lowered if embryos are transferred to the uterine horn contralateral to the corpus luteum. Recipients should be rejected if no corpus luteum is present or pathology of the reproductive tract is noted. The next step is to pass the embryo transfer device, e.g., a standard Cassou inseminating gun, through the cervix. The third step of non-surgical transfer is to insert the tip of the instrument into the desired uterine horn ipsilateral to the corpus luteum. The final step of the procedure is to transfer the embryo from a container, such as a straw, into the desired uterine horn using the transfer device.

Collection of Amniotic or Allantoic Fluid after Embryo Transfer

Certain embodiments of the invention encompass methods of collecting amniotic fluid or allantoic fluid from an embryonic or fetal allantois subsequent to transferring an embryo of the invention into a recipient female. Once amniotic or allantoic fluid is collected, a further aspect includes isolating embryonic or fetal cells from the fluid and performing genomic or other omics-related analyses on DNA or other material extracted from the embryonic or fetal cells or from the amniotic or allantoic fluid itself. A specific embodiment includes extracting embryonic or fetal cell-free DNA from the allantoic fluid and performing genomic or other omics-related analysis on the DNA. Any method known in the art for collection of allantoic fluid may be used in the invention, including but not limited to trans-vaginal/trans-uterine collection using either ultrasound guided or manual puncture techniques. Additionally, allantoic fluid may be collected at any time during gestation in a mother or embryo transfer recipient, including but not limited to within 40, 50 or 60 days of the embryo or fetus's conception or 28 to 60, 30 to 40, 30 to 50, 35 to 40, 35 to 45 or 30 to 35 days after the embryo's or fetus's conception. Techniques for the collection of amniotic and allantoic fluid are described in PCT/US2023/017297 and PCT/US2016/057115, respectively, the disclosures of which are hereby incorporated by reference herein in their entirety.

Deriving Gametes from Embryos

One aspect of the invention comprises deriving gametes, both oocytes and sperm, directly from in vitro, or in vivo, embryos of the invention. In addition to markedly reducing the maturation age of selection candidates, the invention allows a breeder to greatly reduce or entirely eliminate the need for maintaining and caring for young and adult animals as either selection candidates (i.e., prospective parents of the next generation) or as recipients for embryo transfer. In a particular embodiment of the invention, oocytes or sperm can be derived from either embryonic stem cells (ESCs) or from induced pluripotent stem cells (iPSCs) obtained from an embryo of the invention (see, e.g., Hayashi et al., Cell 146, 519-532, 2011; ESC derivation from blastocysts is described in Hayashi et al., Cell 146, 519-532, 2011 and iPSCs derivation from embryonic fibroblasts is described in Hikabe et al., Nature 539, 299-303, 2016; derivation of bovine iPSCs from bovine fetal fibroblasts is described in Talluri et al., Cellular Reprogramming, 17, 131-140, 2015). Once ESCs or iPSCs are derived, they can in turn be used to derive primordial germ cell-like cells (PGCLCs) (see, e.g., Hayashi et al., Cell 146, 519-532, 2011). Finally, PGCLCs can be used to derive sperm (see, e.g., Hayashi et al., Cell 146, 519-532, 2011) or oocytes (see e.g., Hikabe et al., Nature 539, 299-303, 2016), in vitro. In an alternative embodiment, oocytes can be derived from primordial germ cells (PGCs) obtained from an embryo or fetus in accordance with any of the methods known in the prior art (see, e.g., Morohaku et al., PNAS, 113, 9121-9036, 2016).

### Generating Nuclear Transfer Eggs Via Haploidization

Another aspect of the invention comprises generating nuclear transfer eggs using a process of haploidization (see, e.g., Lee, Y., Trout, A., Marti-Gutierrez, N. et al. “Haploidy in somatic cells is induced by mature oocytes in mice,” Commun Biol 5, 95 (2022) (“Lee et al.”)). Haploidization comprises transferring the nucleus of a diploid cell, such as a somatic cell, stem cell, pluripotent stem cell or primordial germ cell—obtained from an embryo of the invention, for example—into an enucleated egg to produce a nuclear transfer egg, which is capable of producing a viable diploid zygote when fertilized with a sperm cell. Any haploidization protocol may be used in the invention, including but not limited to the haploidization protocol described in any of Lee et al; Palermo, G. et al., “Oocyte-induced haploidization,” Reprod BioMed Online vol. 4, no. 3:237-242 (2002); and Tesarik, J. et al., “Fertilizable oocytes reconstructed from patient's somatic cell nuclei and donor ooplasts,” Reprod BioMed Online vol. 2, no. 3:237-242 (2001). Using the process of haploidization described by those authors the replacement of meiotic spindles in a mature or immature oocyte with the nucleus of one of the aforementioned donor cell types results in the formation of de novo spindles consisting of donor cell homologous chromosomes comprised of single chromatids. Fertilization of such a nuclear transfer egg with a sperm cell results in the extrusion of one set of homologous chromosomes into the pseudo-polar body, resulting in a viable zygote with haploid somatic and sperm pronuclei. (See Lee et al., abstract.)

Use of Gametes Derived from Embryos and Nuclear Transfer Eggs in Assisted Reproductive Technologies

In certain embodiments of the invention, once gametes are derived from one or more embryos, or nuclear transfer eggs are generated from one or more embryos, those gametes are used in IVF, or other assisted reproductive technologies such as intracytoplasmic sperm injection, to generate a subsequent generation of embryos. In a further embodiment, this process is repeated multiple times, with each set of derived gametes or nuclear transfer eggs used to generate a subsequent generation of embryos.

### Cloning

An additional aspect of the invention encompasses cloning embryos that have been genomically evaluated using the techniques disclosed herein, either before or after transfer into a recipient. Cloning is generally understood to be the creation of a living animal/organism that is essentially genetically identical to the unit or individual from which it was produced. In those embodiments of the invention that encompass cloned embryos, any method by which an animal can be cloned that is known in the art can be utilized. Thus, it is contemplated that cloned embryos and cloned fetuses are produced by any conventional method, for instance including the cloning techniques described herein, as well as those described in international patent application PCT/US01/41561. In one aspect of the invention, a basis for cloning an embryo or a fetus is its genomic merit. In a further aspect, the embryo's genetic merit is determined by genomic analysis as disclosed herein.

Cloning of embryos by nuclear transfer has been developed in several species. This technique generally involves the transfer of a cell nucleus (obtained from a donor cell) into an enucleated cell, for instance, a metaphase II oocyte. This oocyte has the ability to incorporate the transferred nucleus and support development of a new embryo (Prather et al., Biol. Reprod 41:414-418, 1989; Campbell et al., Nature 380:64-66, 1996; Wilmut et al., Nature 385:810-813, 1997). Morphological indications of this re-programming are the dispersion of nucleoli (Szollosi et al., J. Cell Sci. 91:603-613, 1988) and swelling of the transferred nucleus (Czolowska et al., 1984; Stice and Robl, Biol. Reprod 39:657-664, 1988; Prather et al., J. Exp. Zool. 225:355-358, 1990; Collas and Robl. Biol. Reprod 45:455-465, 1991). The most conclusive evidence that the oocyte cytoplasm has the ability to re-program is the birth of offspring from nuclear transplant embryos in several species, including sheep (Smith and Wilmut, Biol. Reprod. 40:1027 1035, 1989; Campbell et al., Nature 380:64-66, 1996; Wells et al., Biol. Reprod. 57:385-393, 1997), cattle (Wells et al., Biol. Reprod. 60:996-1005, 1999; Kato et al., Science 282:2095-2098, 1998; Prather et al., Biol. Reprod. 37:859-866, 1987; Bondioli et al., Theriogenology 33:165-174, 1990), pigs (Prather et al., Biol. Reprod. 41:414-418, 1989) and rabbits (Stice and Robl, Biol. Reprod. 39:657-664, 1988).

### EXAMPLE 1

The purpose of this Example was to find a use for bovine IVF embryos having a quality score of “Code 2,” which, within the breeding program in question, are not transferred into the recipients and are instead discarded. Normally the pregnancy rate for bovine IVF embryos with a quality score of Code 2 is around 30 to 35%, but these embryos have a high rate of pregnancy loss, having pregnancy rates at 60 days of around 25%.

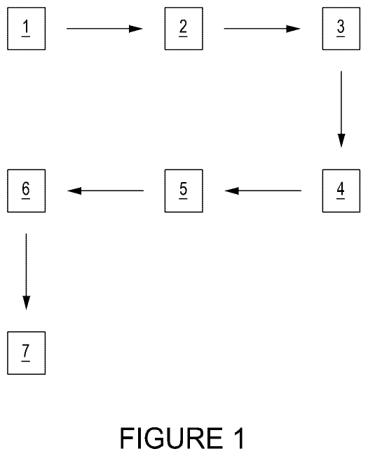

FIG. 1 shows steps involved in utilizing Code 2 quality embryos, as in this Example. Referring to FIG. 1, the first step 1 involves identifying Code 2 quality embryos from among a larger population of embryos. In the second step 2, the identified Code 2 quality embryos are each cultured using the techniques described in the section entitled “Generating Pluripotent Stem Cell Cultures from Unselected Embryos,” above. In the third step 3, after approximately 4 to 11 days of culture, cell samples are obtained from each culture and genomically evaluated. In the fourth step 4, cultures yielding low genomic values are discarded, while cultures yielding high genomic values are maintained. In the fifth step 5, cell samples from cultures yielding high genomic values are frozen. In the sixth step 6, donor cells for use in cloning are obtained from these frozen samples. In the seventh step 7, the donor cells are used to produce clones (via somatic cell nuclear transfer, for example).

In Example 1, forty-five bovine IVF embryos (quality score of Code 2) were put in culture on Day 10 as described in the section entitled “Generating Pluripotent Stem Cell Cultures from Unselected Embryos,” above. Specifically, each embryo was placed into an individual well of a 12 well tissue culture-treated dish (Falcon) coated with irradiated CF1 Mouse Embryonic Fibroblasts (MEFs) (Gibco) and cultured with a serum-free stem cell media (Stem Cell Technologies, Canada) as base media, supplemented with (13.4 mg/mL) Low Free Fatty Acid BSA (MP Biomedicals NZ), 20 ng/mL bovine FGF2 (bFGF2) (Peprotech, USA), 2.5 μM IWR1 (Sigma, USA) and 1×penicillin-streptomycin (Gibco), at 37° C. and 5% CO₂. After 72 hours, the culture media was changed for the first time, and the embryos that did not attach to the feeder layer, were mechanically pressed against the bottom of the well with a 21 G needle, then the media was changed daily. After 4 to 11 days of culture, the outgrowths were dissociated manually and passed to a new well, previously coated with MEFs, and cultured in the same media supplemented with 10 μM Y-27632 (Enzo life science). 27 cell samples were sent for genomic evaluation on the day of the first passage. Based on the results of these genomic evaluations, two cell lines (Total Performance Index (TPI) around 2900) were cryopreserved for later use (e.g., in cloning)—one of them with above average cell proliferation and a second one with normal cell proliferation. Two others cell lines were discarded based on the results of the genomic evaluation (TPI around 2710).

### EXAMPLE 2

23 bovine IVF embryos (quality score of Code 2) were put in culture using the techniques described in the section entitled “Generating Pluripotent Stem Cell Cultures from Unselected Embryos” and Example 1, above. Cell cultures were successfully generated from 19 of the 23 embryos, and cell samples from 18 of these cell cultures were sent for genomic evaluation. 8 ESC lines were ultimately established based on the obtained genomic evaluations.

### EXAMPLE 3

Using the above-described technique for establishing a pluripotent stem cell culture, 4 vitrified bovine clones were produced from stem cells generated from an IFV embryo with a quality score of Code 2. The bovine clones were subsequently transferred into recipient females, with one of the clones resulting in the birth of a calf.

Although the foregoing invention has been described in some detail, one of ordinary skill in the art will understand that certain changes and modifications may be practiced within the scope of the claims.

What we claim is:

1. A method of establishing a stem cell culture from a non-human mammalian embryo comprising
a) generating a population of embryos, in vitro or in vivo;
b) assessing the morphology of each of the embryos in the population;
c) selecting a subpopulation of embryos for embryo transfer based on the morphological assessment, thereby establishing a group of selected embryos and a group of unselected embryos in the population; and
d) establishing a stem cell culture from an embryo in the group of unselected embryos.

2. The method of claim 1, further comprising the step of obtaining omics data from one or more cells from the stem cell culture.

3. The method of claim 1, further comprising the step of extracting DNA from one or more cells from the stem cell culture.

4. The method of claim 3, further comprising the steps of genotyping the DNA extracted from the one or more cells from the stem cell culture to obtain a genotype and determining a genomic estimated breeding value (GEBV) or a genomic predicted transmitting ability (GPTA) based on the obtained genotype.

5. The method of claim 1, further comprising the step of producing a clone or a nuclear transfer egg using a cell from the stem cell culture.

6. The method of claim 5, further comprising the step of transferring the clone into a recipient animal.

7. The method of claim 6, further comprising the step of obtaining one or more embryonic or fetal cells from within an allantois of the transferred clone.

8. The method of claim 7, further comprising the step of extracting DNA from the one or more embryonic or fetal cells.

9. The method of claim 8, further comprising the step of genotyping the DNA extracted from the one or more embryonic or fetal cells to obtain a genotype for the clone.

10. The method of claim 9, further comprising the step of determining a genomic estimated breeding value (GEBV) or a genomic predicted transmitting ability (GPTA) of the clone using the genotype.

11. The method of claim 10, further comprising the step of selecting the transferred clone as a parent or for production of gametes based on the determined GEBV or GPTA.
